(19) United States
(12) Patent Application Publication
(10) Pub. No.: US 2023/0307145 A1
(43) Pub. Date: Sep. 28, 2023

(54) SIGNAL PROCESSING APPARATUS, SIGNAL PROCESSING METHOD, AND NON-TRANSITORY COMPUTER READABLE MEDIUM

(71) Applicant: Hitachi, Ltd., Tokyo (JP)

(72) Inventors: Takuma SHIBAHARA, Tokyo (JP); Yasuho YAMASHITA, Tokyo (JP); Shota NEMOTO, Tokyo (JP)

(21) Appl. No.: 18/096,155

(22) Filed: Jan. 12, 2023

(30) Foreign Application Priority Data
Mar. 25, 2022 (JP) 2022-050434

(51) Int. Cl. G16H 50/70

(52) U.S. Cl. CPC G16H 50/70

(57) ABSTRACT

Signal processing assists in searching for a mechanism through a generation equation of a signal for stratifying a patient, which refers to classification of patients suffering from a disease to enable medical treatment. The signal processing includes: storing analysis target data that includes values of an explanatory variable and an objective variable; retaining action(s) which are either the explanatory variable or a modulation method for modulating the explanatory variable; generating, based on the action history information, a first signal obtained by modulating the analysis target data; generating a first multispectral signal obtained by classifying the first signal for each analysis target into a first spectral signal for each value of the objective variable; generating, based on the first multispectral signal, a signal distribution obtained by one-dimensionally arranging a distribution of the first signal based on the value of the objective variable, and outputting the signal distribution in a displayable manner.

### CLAIM OF PRIORITY

The present application claims priority from Japanese patent application No. 2022-50434 filed on Mar. 25, 2022, the content of which is hereby incorporated by reference into this application.

### BACKGROUND OF THE INVENTION

### 1. Field of the Invention

The present invention relates to a signal processing apparatus, a signal processing method, and a non-transitory computer readable medium.

### 2. Description of the Related Art

In medical terms, patient stratification refers to classification of patients suffering from a disease by using the patient and disease-specific biometric information (blood, genetic information, etc.) to enable individual medical treatment. The patient stratification allows physicians to quickly and accurately determine whether to administer drugs to individual patients. Therefore, the patient stratification contributes to rapid recovery of individual patients and leads to reduction in accelerating increase in medical costs, which serves both interests of individuals and society as a whole.

In addition, Subrahmanyam, Priyanka B., et al. “Distinct predictive biomarker candidates for response to anti-CTLA-4 and anti-PD-1 immunotherapy in melanoma patients.” Journal for immunotherapy of cancer 6, Article number: 18 (2018), Published: 6 Mar. 2018 (Non-patent Literature 1) discloses a method for stratifying skin cancer (melanoma) patients according to characteristics of immune cells. Volodymyr Mnih, Koray Kavukcuoglu, et al. “Playing atari with deep reinforcement learning.” arXiv preprint arXiv: 1312.5602 (2013), Published: 19 Dec. 2013 (Non-Patent Literature 2) discloses a configuration that handles a multispectral image (color image).

Non-patent Literature 1 discloses the method for stratifying skin cancer (melanoma) patients according to characteristics of immune cells. At this time, distributions of 40 types of immune cells shown in Table 3 are visualized as images by a viSNE method (see b and c in FIG. 1). By visually comparing the images, it is possible to stratify a group of patients (efficacy group) for which a drug is effective and a group of patients (non-efficacy group) for which the drug is not effective.

The method of Non-patent Literature 1 may not lead to specification of factors because the method is a complicated visual confirmation operation. In addition, in the case of a drug for which an efficacy group and a non-efficacy group are stratified by a combination of a plurality of factors, it is significantly difficult to visually find the combination from the visualized image shown in c of FIG. 1 of Non-Patent Document 1. In particular, it is not clear what a vertical axis and a horizontal axis of b and c in FIG. 1 converted by the viSNE method mean medically. Performing treatment based on a value whose mechanism is unknown is a factor in lowering the reliability of treatment.

### SUMMARY OF THE INVENTION

An object of the invention is to assist in searching for a mechanism through a generation equation of a signal for stratifying a patient.

A signal processing apparatus according to an aspect of the invention disclosed in the present application includes: a storage unit configured to store an analysis target data group including, for each analysis target, analysis target data that includes a value of an explanatory variable and a value of an objective variable for the analysis target, and action history information retaining one or more actions which are either the explanatory variable or a modulation method for modulating the explanatory variable; a modulation unit configured to generate, based on the action history information, a first signal obtained by modulating the analysis target data for each analysis target; a generation unit configured to generate a first multispectral signal obtained by classifying the first signal modulated by the modulation unit for each analysis target into a first spectral signal for each value of the objective variable; and an output unit configured to generate, based on the first multispectral signal, a signal distribution obtained by one-dimensionally arranging a distribution of the first signal based on the value of the objective variable, and output the signal distribution in a displayable manner.

According to representative embodiments of the invention, it is possible to assist in searching for a mechanism through a generation equation of a signal for stratifying a patient. Problems, configurations, and effects other than those described above are made clear by the following description of the embodiments.

### BRIEF DESCRIPTION OF THE DRAWINGS

FIG. 1 is a block diagram showing a hardware configuration example of a signal processing apparatus.

FIG. 2 is a diagram showing an example of an analysis target DB.

FIG. 3 is a diagram showing an example of a pattern DB.

FIG. 4 is a block diagram showing a circuit configuration example of a signal processing circuit.

FIG. 5 is a block diagram showing a configuration example of a controller.

FIG. 6 is a flowchart showing a main routine as an example of a processing procedure performed by a signal processing apparatus according to a first embodiment.

FIG. 7 is a diagram showing an example of a display screen according to the first embodiment.

FIG. 8 is a diagram showing an example of action history information.

FIG. 9 is a flowchart showing an example of a detailed processing procedure of a subroutine in the main routine in step S602.

FIG. 10 is a diagram showing an example of a multispectral signal according to the first embodiment.

FIGS. 11(A) to 11(C) are diagrams showing a calculation example of Overwrap and Margin according to the first embodiment and FIG. 11(D) is a legend for FIGS. 11(A) to 11(C).

FIG. 12 is a diagram showing an example of patient data used in an operational experiment of the signal processing apparatus according to the first embodiment.

FIG. 13 is a graph showing a first example of an operation experiment result of the signal processing apparatus according to the first embodiment.

FIG. 14 is a graph showing a second example of the operation experiment result of the signal processing apparatus according to the first embodiment.

FIG. 15 is a flowchart showing a main routine as an example of a processing procedure performed by a signal processing apparatus according to a second embodiment.

FIG. 16 is a diagram showing an example of a display screen according to the second embodiment.

FIG. 17 is a flowchart showing an example of a detailed processing procedure of a subroutine 1700 in the main routine in step S1502.

FIG. 18 is a diagram showing an example of a multispectral signal S(t) according to the second embodiment.

FIGS. 19(A) to 19(D) are diagrams showing a visualization example of the multispectral signal S(t) according to the second embodiment and FIG. 19(E) is a legend for FIGS. 19(A) to 19(D).

### DESCRIPTION OF EMBODIMENTS

### First Embodiment

Hereinafter, an example of a signal processing apparatus, a signal processing method, and a non-transitory computer readable medium according to a first embodiment will be described with reference to the accompanying drawings. In the first embodiment, a data group to be analyzed is, for example, a set of analysis target data sets, each of which is a combination of an objective variable indicating a health condition and analysis target data indicating, as an explanatory variable, 100 types of patient information including a weight and a height, for each of 50 diabetes patients. Note that the number of patients and the number of types of patient information are examples.

### Hardware Configuration Example of Signal Processing Apparatus

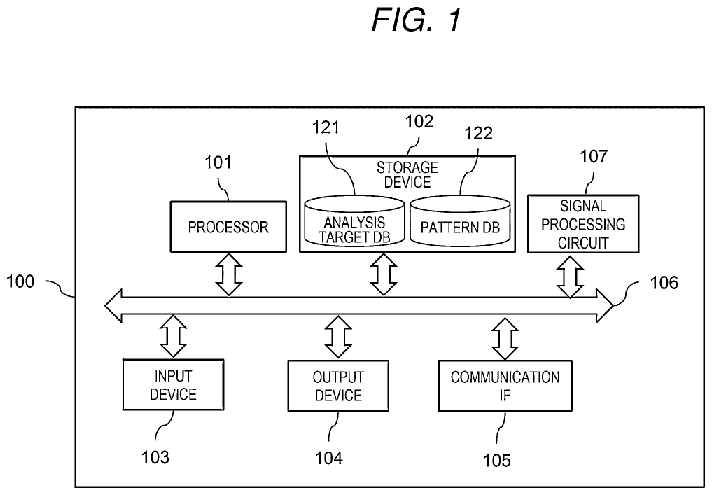

FIG. 1 is a block diagram showing a hardware configuration example of the signal processing apparatus. A signal processing apparatus 100 includes a processor 101, a storage device 102, an input device 103, an output device 104, a communication interface (IF) 105, a bus 106, and a signal processing circuit 107. The processor 101, the storage device 102, the input device 103, the output device 104, the communication IF 105, and the signal processing circuit 107 are connected by the bus 106.

The processor 101 controls the signal processing apparatus 100. The storage device 102 serves as an operation area of the processor 101. The storage device 102 is a non-transitory or temporary recording medium that stores various programs and data. Examples of the storage device 102 include a read only memory (ROM), a random access memory (RAM), a hard disk drive (HDD), and a flash memory. The input device 103 inputs data. Examples of the input device 103 include a keyboard, a mouse, a touch panel, a numeric keypad, and a scanner. The output device 104 outputs data. Examples of the output device 104 include a display and a printer. The communication IF 105 is connected to a network and transmits and receives data.

In addition, the signal processing apparatus 100 stores an analysis target data base (DB) 121 and a pattern DB 122 in the storage device 102. Hereinafter, specific description will be made.

### Configuration Example of Analysis Target DB 121

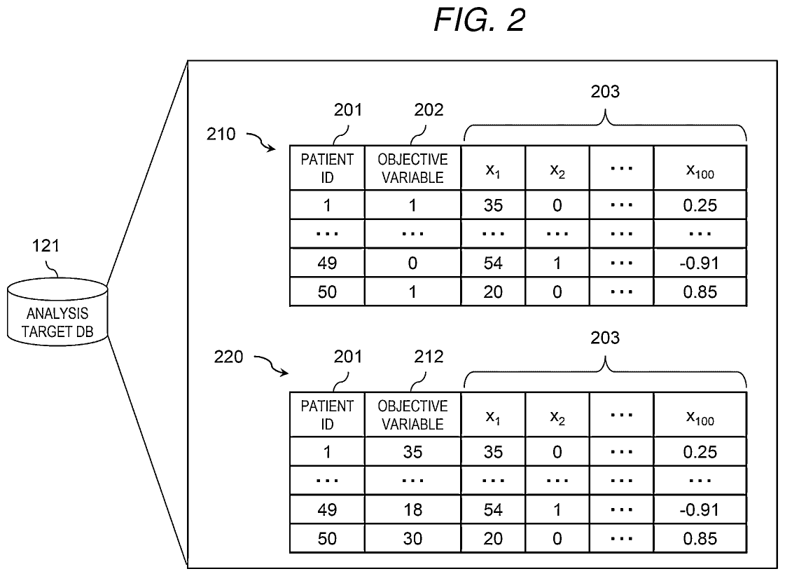

FIG. 2 is a diagram showing an example of the analysis target DB 121. The analysis target DB 121 stores first analysis target data 210 and second analysis target data 220. The second analysis target data 220 is used in a second embodiment, and will be described later.

The first analysis target data 210 includes, as fields, a patient ID 201, an objective variable 202, and an explanatory variable group 203. A combination of values of each field in the same row is an analysis target data set of one patient. The patient ID 201 is identification information for distinguishing a patient, which is an example of an analysis target, from other patients, and the value of the patient ID 201 is represented by, for example, 1 to 50. The objective variable 202 indicates a value indicating a health condition of the patient.

In the first embodiment, a value is stored that indicates whether a body mass index (BMI) exceeds a reference value (1: applicable, 0: non-applicable). Each explanatory variable of the explanatory variable group 203 indicates patient information. In the first embodiment, a total of 100 types of patient information including “x₁: age”, “x₂: sex”, “x₃: height”, and “x₄: weight” are included. For example, a value of the explanatory variable “x₁” in the explanatory variable group 203 is “35” when the patient ID 201 is “1”.

### Configuration Example of Pattern DB 122

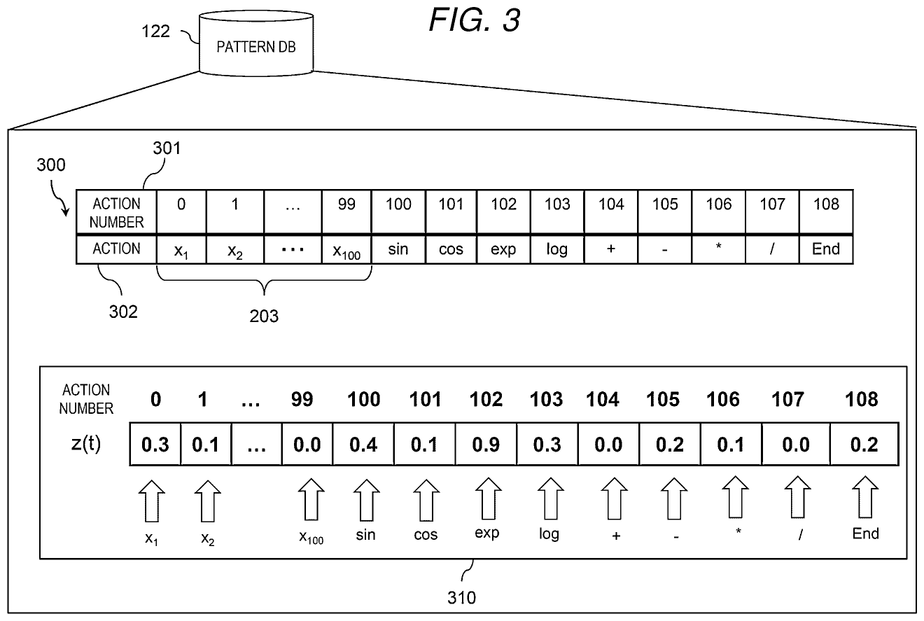

FIG. 3 is a diagram showing an example of the pattern DB 122. The pattern DB 122 stores a pattern table 300 and a value map 310. The pattern table 300 defines the types of control signals for a modulator 401, which will be described later. The contents of the pattern table 300 are set in advance.

The pattern table 300 includes, as fields, an action number row 301 and an action row 302. A numerical value in ascending order from 0 to 108 in each column in the action number row 301 is an action number, which is hereinafter referred to as an action number 301. A value of each column in the action row 302 is an action, which is hereinafter referred to as an action 302.

The action number 301 is an identification number for uniquely specifying the action 302. The action 302 includes explanatory variables x₁, x₂, . . . , and x₁₀₀ of the explanatory variable group 203, operators having the explanatory variables x₁, x₂, . . . , and x₁₀₀ as operands, and an indicator End indicating the end of the operation. The operators include a unary operator and a multiple operator. The unary operator includes, for example, a sin function, a cos function, an exponential function, and a logarithmic function. For example, the multiple operator includes a four arithmetic operator. The value map 310 will be described later.

### Configuration Example of Signal Processing Circuit 107

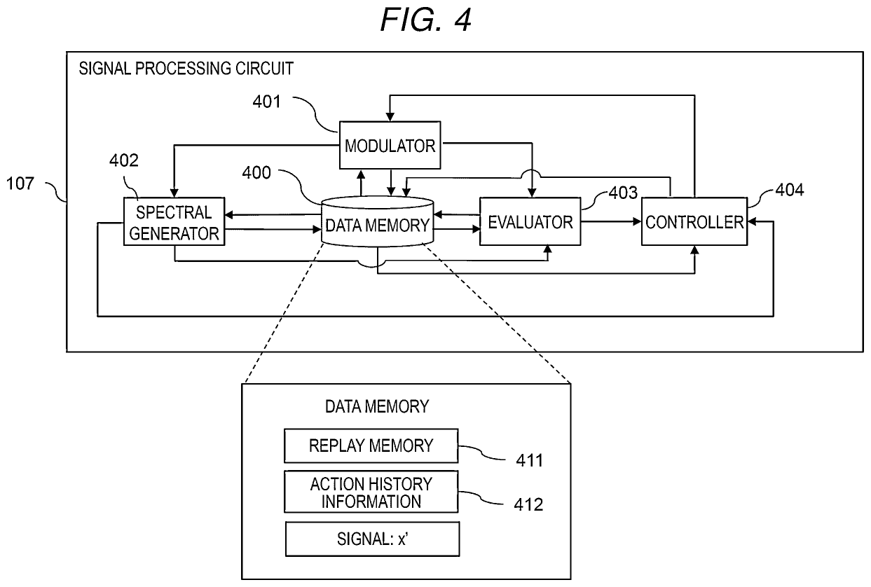

FIG. 4 is a block diagram showing a circuit configuration example of the signal processing circuit 107. The signal processing circuit 107 includes a data memory 400, the modulator 401, a spectral generator 402, an evaluator 403, and a controller 404. Arrows in FIG. 4 represent flows of data generated by the respective units (401 to 404). The signal processing circuit 107 is implemented by a circuit configuration, but may be implemented by causing the processor 101 to execute a program stored in the storage device 102.

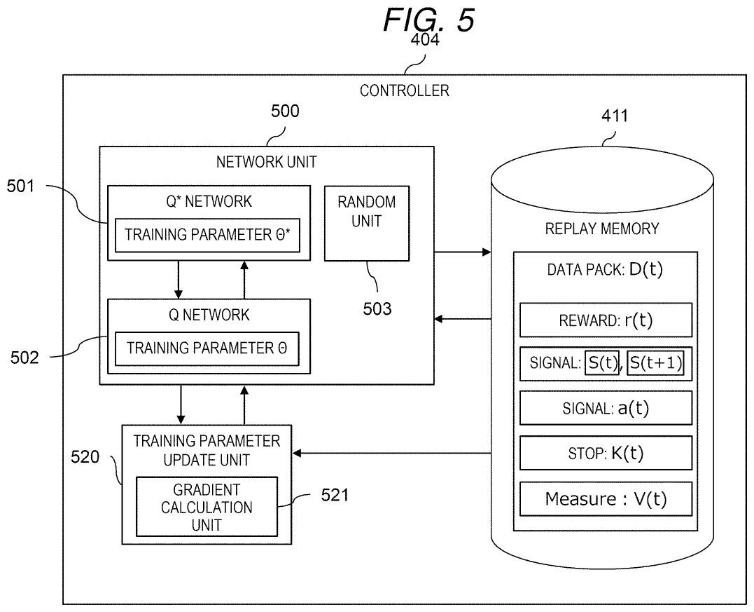

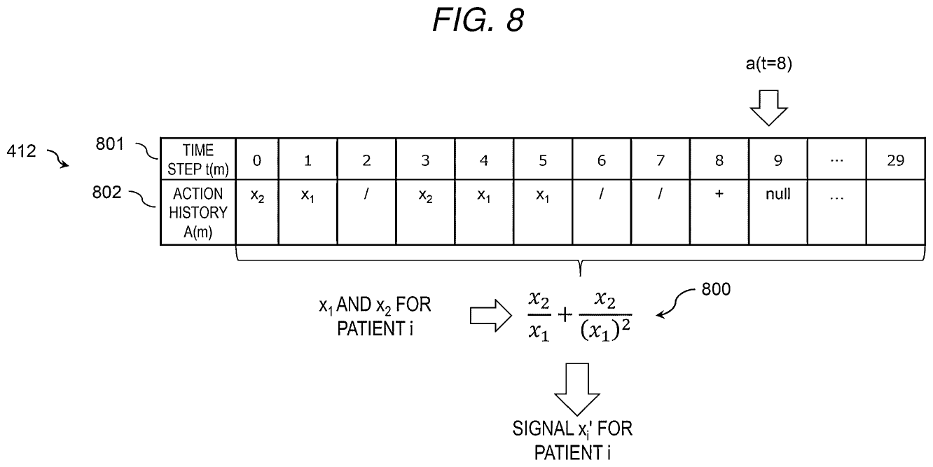

The data memory 400 includes a replay memory 411, action history information 412, and a signal x′. Details of the replay memory 411 will be described later with reference to FIG. 5. Details of the action history information 412 will be described later with reference to FIG. 8. The signal x′ is a numerical value for stratifying patients.

### Configuration Example of Controller 404

FIG. 5 is a block diagram showing a configuration example of the controller 404. The controller 404 includes a network unit 500, the replay memory 411, and a training parameter update unit 520. The network unit 500 includes a Q* network 501, a Q network 502, and a random unit 503. The Q* network 501 and the Q network 502 are value functions of the same configuration that learn an action that maximizes a value called a value. The Q* network 501 includes a training parameter θ*. The Q network 502 includes a training parameter θ. The random unit 503 outputs a random numerical value in a range of, for example, 0.0 to 1.0.

The replay memory 411 stores a data pack D(t). The data pack D(t) includes a reward r(t), multispectral signals S(t) and S(t+1), a control signal a(t), a stop signal K(t), and a statistic V(t) at a time step t. When the action 302 (the control signal a(t)) is taken in the state of the time step t (in the case of the multispectral signal S(t)), the data pack D(t) specifies whether to reset an action history row 802 and the time step t (the stop signal K(t)).

The training parameter update unit 520 includes a gradient calculation unit 521. The training parameter update unit 520 calculates, using the gradient calculation unit 521, a gradient g in consideration of the reward r(t), and updates the training parameter θ by adding the gradient g to the training parameter θ. The controller 404 is implemented by a circuit configuration, but may be implemented by causing the processor 101 to execute a program stored in the storage device 102.

### Example of Processing Procedure

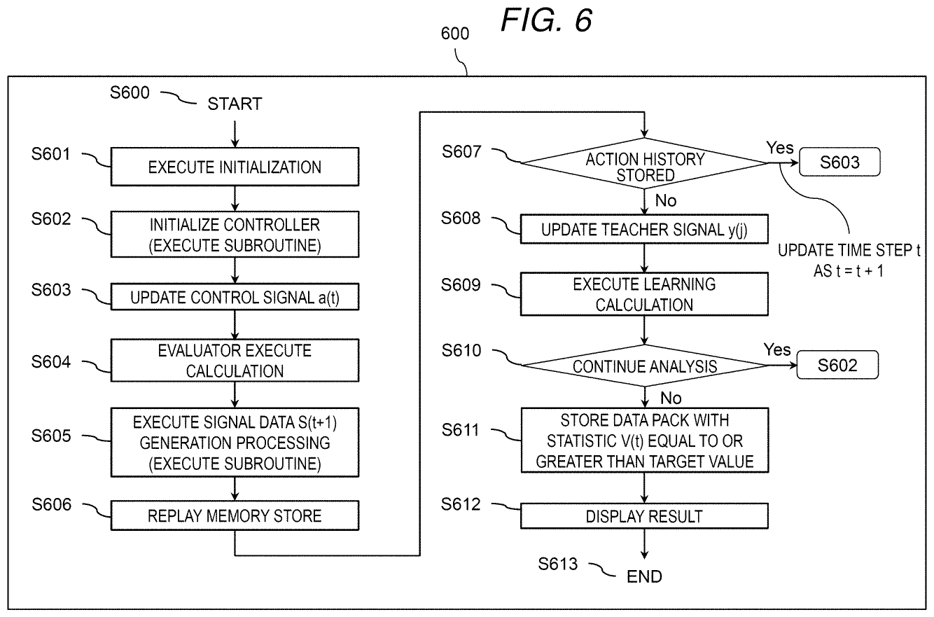

FIG. 6 is a flowchart showing a main routine 600 as an example of a processing procedure performed by the signal processing apparatus 100 according to the first embodiment. Hereinafter, the flow of the processing of the main routine 600 will be described with reference to the flowchart of FIG. 6.

### Step S600

A display screen is displayed on the output device 104.

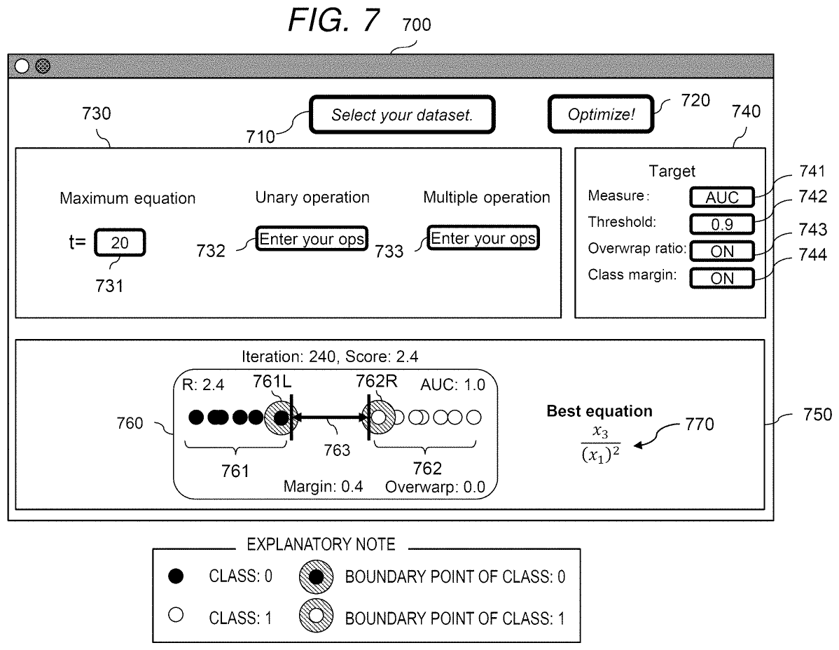

FIG. 7 is a diagram showing an example of the display screen according to the first embodiment. A display screen 700 includes a load button 710, a start button 720, a generation condition input area 730, a target scale input area 740, and a result display area 750.

The load button 710 is a user interface for loading the first analysis target data 210 in the analysis target DB 121 and the pattern table 300 in the pattern DB 122. In step S600, when the load button 710 is clicked by an operation of a user, the processor 101 loads the first analysis target data 210 in the analysis target DB 121 and the pattern table 300 in the pattern DB 122, which are stored in the storage device 102, by using a function of an operation system. Then, the processor 101 transfers the first analysis target data 210 and the pattern table 300 to the data memory 400 of the signal processing circuit 107.

The start button 720 is a user interface for the signal processing apparatus 100 to start processing. When the start button 720 is clicked by the operation of the user, the process is started from step S601.

The generation condition input area 730 is an area for receiving an input of a generation condition of an expression, and specifically includes, for example, an expression length input area 731, a unary operator input area 732, and a multiple operator input area 733.

The expression length input area 731 is an input field for receiving an upper limit value input of a length of an expression to be generated. When the expression length input area 731 is blank, a numerical value of a default maximum expression length (30 in this example) is automatically set.

The unary operator input area 732 is an input field for receiving an additional input of a unary operator, which is one of modulation methods in the modulator 401. Examples of the unary operator that can be additionally input to the unary operator input area 732 include a hyperbolic function and a constant multiplication function that are not registered in the pattern table 300. When no additional input is made, the unary operator (sin function, cos function, exponential function, logarithmic function) registered in the pattern table 300 is applied.

The multiple operator input area 733 is an input field for receiving an additional input of a multiple operator, which is one of the modulation methods in the modulator 401. Examples of the multiple operator that can be additionally input to the multiple operator input area 733 include a max function and a min function that are not registered in the pattern table 300. When no additional input is made, the multiple operator (+, −, ×, /) registered in the pattern table 300 is applied.

The target scale input area 740 is an area for receiving an input of a target scale by the operation of the user. Specifically, for example, the target scale input area 740 includes a statistic selection unit (Measure) 741, a target value setting unit (Threshold) 742, an overlap ratio selection unit (Overwrap ratio) 743, and an inter-class margin selection unit (Class margin) 744.

The statistic selection unit 741 is a user interface for the user to select the statistic V(t) (for example, accuracy, precision, recall, or f-measure) for evaluating prediction accuracy of an identification model. In FIG. 7, “AUC” (accuracy) is selected as the statistic V(t) in order to determine whether efficacy or non-efficacy is good or bad.

The target value setting unit 742 is a user interface for receiving a target value input of the statistic V(t) selected by the statistic selection unit 741. In FIG. 7, “0.9” is input as the target value.

The overlap ratio selection unit 743 is a user interface for selecting whether to incorporate, as a score, a ratio at which signal values of different classes have the same value, and either ON (incorporation) or OFF (non-incorporation) is selected. In FIG. 7, ON is set in the overlap ratio selection unit 743.

The inter-class margin selection unit 744 is a user interface for selecting whether to incorporate as a margin between different classes, and either ON (incorporation) or OFF (non-incorporation) is selected. In FIG. 7, ON is set in the inter-class margin selection unit 744.

The result display area 750 is an area for displaying a processing result by the signal processing apparatus 100. Specifically, for example, the result display area 750 includes a signal distribution 760 and a generation equation 770. The signal distribution 760 is a graphic user interface indicating a one-dimensional distribution of a set of points (• and ∘) corresponding to the patients. In the example of FIG. 7, a patient group is classified into two classes (a class 0 and a class 1), a set of points (•) corresponding to patients belonging to the class 0 is a point group 761 of the class 0, and a set of points (∘) corresponding to patients belonging to the class 1 is a point group 762 of the class 1.

Further, a position of each point (• and ∘) is a value calculated by the generation equation 770 as a result of substituting, into the generation equation 770, the value of the explanatory variable existing in the generation equation 770 among the values of the explanatory variable group 203 of a patient corresponding to the point, i.e., the signal x′. The larger the calculated value is, the more the point is located on a right side, and the smaller the calculated value is, the more the point is located on a left side.

A point 761L at a left end of the point group 761 of the class 0 is a boundary point 761L of the class 0, and corresponds to the patient having the maximum calculated value in the point group 761 of the class 0. A point 762R at a right end of the point group 762 of the class 1 is a boundary point 762R of the class 1, and corresponds to the patient having the minimum calculated value in the point group 762 of the class 1. A margin 763 is an interval between the boundary point 761L and the boundary point 762R, i.e., a difference between the calculated values.

The generation equation 770 is an equation for implementing stratification, which is easily handled by a doctor or a researcher, in the signal distribution 760, and is generated by the signal processing apparatus 100. A method for generating the generation equation 770 will be described later.

When the start button 720 is clicked by the operation of the user, the process is started from step S601.

### Step S601

Returning to FIG. 6, the signal processing apparatus 100 initializes a calculation step m to m=0. The Q* network 501 and the Q network 502 are value functions of the same configuration that learn the control signal a(t), which is the action 302 that maximizes a value called a value. The value in this case is an amount by which the control signal a(t) affects the reward r(t). The control signal a(t) that increases the reward r(t) is of a high value.

Here, the value map 310 shown in FIG. 3 will be described in detail. The value map 310 represents the value in each action 302 in the pattern table 300 when a certain action 302 (a certain control signal a(t)) is taken in a certain state (in the case of a certain multispectral signal S(t)). The value map 310 at the time step t is referred to as a value map z(t).

When the multispectral signal S(t) is input, the Q network 502 and the Q* network 501 calculate the value map 310 and select the action 302 corresponding to the action number 301 having the maximum value in the value map 310. In the example of FIG. 3, since the maximum value is “0.9”, “exp” (exponential function) with the value of the action number 301 of “102” is selected as the action 302.

The Q network 502 and the Q* network 501 in the first embodiment can output the value map 310. As a specific calculation method of the value map 310, deep reinforcement learning, i.e., deep Q-network (DQN) as shown in Non-patent Literature 2 can be applied.

A configuration example of the network 501 in the case of the multispectral signal S(t) in the first embodiment will be specifically described. The Q* network 501 will be described by taking, for example, a case where the multispectral signal S(t), which is a set of 84-dimensional spectral signals, is input as an example. In the first embodiment, the multispectral signal S(t) includes two types of spectral signals (spectral signals of two classes of 0 and 1).

Here, a configuration example of the Q* network 501 will be described. A first layer of the Q* network 501 is a convolutional network (kernel (neuron): 8 signals, stride: 4, activation function: ReLU). A second layer of the Q* network 501 is a convolutional network (kernel (neuron): 4 signals, stride: 2, activation function: ReLU). A third layer of the Q* network 501 is a fully-connected network (number of neurons: 256, activation function: ReLU).

An output layer of the Q* network 501 is a fully-connected network, and outputs z(t) as the value map 310 corresponding to the action row 302 of the pattern table 300. The value map z(t) corresponds to each action 302 of the pattern table 300 on a one-to-one basis. That is, the value map z(t) is an array having values corresponding to 109 actions 302.

The training parameters θ* of the Q* network 501 are neurons (i.e., real-valued matrices) from the first layer to the third layer of the Q* network 501. The Q network 502 has the same configuration as the Q* network 501. As described above, the Q network 502 and the Q* network 501 can calculate the value map z(t) using the multispectral signal S(t) as an input and select, from the pattern table 300, the action 302 corresponding to the action number 301 having the maximum value.

Returning to FIG. 6, in step S601, the signal processing apparatus 100 initializes the training parameter θ* of the Q* network 501 with the random numerical value of the random unit 503, and initializes the training parameter θ of the Q network 502 with the random numerical value of the random unit 503.

Here, an effect obtained when the multispectral signal S(t) is handled in the Q network 502 and the Q* network 501 will be described. In the signal processing apparatus 100, a memory amount on the computer occupied by the multispectral signal S(t) is O (n²). On the other hand, in the case of a multispectral image (i.e., a color image based on three kinds of RGB spectra) as shown in Non-Patent Literature 2, a memory amount on the computer occupied by one image is O (n³).

In the first embodiment, when a signal length n of the spectral signal is set to 84 (i.e., 84 dimensions), the memory amount can be simply 84 times smaller and a capacity of the replay memory 411 can be reduced to 1/n by handling the multispectral signal S(t). In addition, by using the multispectral signal S(t), a communication speed between the network unit 500 and the training parameter update unit 520 and the replay memory 411 can be improved by n times in the controller 404.

When the controller 404 causes the processor 101 to execute the program stored in the storage device 102, the communication performed on the bus 106 is also improved by n times. On the other hand, an information amount of the input data used when the Q network 502 calculates the value map 310 is 1/n of an information amount of the multispectral image.

At this time, there is a concern that the calculation of the value map 310 is correctly performed. However, in the first embodiment, by generating the multispectral signal S(t) using a subroutine 900 to be described later, the value map 310 is accurately generated, and the generation equation 770 can be obtained that implements stratification which is easy for the doctor or the researcher to handle.

### Step S602

The signal processing apparatus 100 initializes the controller 404. Specifically, for example, the signal processing apparatus 100 sets the action history information 412 to an initial state, and executes a subroutine in the main routine 600.

FIG. 8 is a diagram showing an example of the action history information 412. The action history information 412 includes a time step row 801 and the action history row 802. The time step row 801 is a time-series time step t(m) at the calculation step m. A numerical value in ascending order of 0, 1, 2, . . . , and 29 in a column in the time step row 801 is the time step t(m). The action history row 802 is an action history A(m) serving as sequence data of the time-series action 302 corresponding to the time step t(m). A value (x₂, x₁, /, and . . . ) in a column in the action history row 802 is the action 302 at the time step t(m).

In step S602, the signal processing apparatus 100 sets the time step t of the time step row 801 to t=0, and sets the action history information 412 to an initial state by leaving all columns in the action history row 802 blank. Then, the signal processing circuit 107 executes a subroutine to calculate the multispectral signal S(t=0) and the signal x′. An expression 800 at the end of the main routine 600 is the generation equation 770 shown in FIG. 7.

### Subroutine

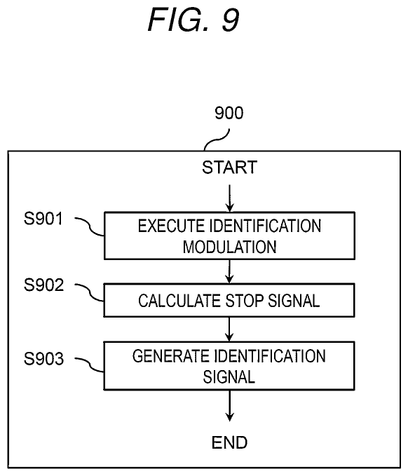

FIG. 9 is a flowchart showing an example of a detailed processing procedure of the subroutine in the main routine 600 in step S602. The subroutine 900 is called and executed by step S602 and step S605 of the main routine 600.

### Step S901

The modulator 401 executes identification modulation. Specifically, for example, the modulator 401 selects an explanatory variable or a modulation method from the control signal a(t) output from the controller 404 at the time step t (t is an integer of 0 or more and T−1 or less, and T is a total number of steps at the time step t, for example T=30). The modulator 401 may receive selection of the explanatory variable or the modulation method selected by the user.

Next, the modulator 401 adds the selected explanatory variable or modulation method to the column in the action history row 802 at the time step t. The action history information 412 is sequence data with columns in the actions 302 at time steps t=0 to T−1. An initial value of the action history row 802 is blank for all columns, as described in step S602.

When reading out the sequence data indicated by the action history row 802 column by column in ascending order of the time step t, the modulator 401 generates an expression by a reverse polish notation. In the example of FIG. 8, the expression 800 is generated.

The modulator 401 may use an expression notation other than the reverse polish notation, for example, a polish notation or an infix notation. In the case of the infix notation, “(“ ”)” are added to the pattern table 300 as the type of operation.

A calculation example of the signal x′ by the expression 800 will be described. When the expression 800 is generated, the modulator 401 substitutes, into the expression 800, the value of the explanatory variable existing in the expression 800 from the explanatory variable group 203 of a patient (hereinafter, referred to as patient i) whose value of the patient ID 201 is i (i is an integer), thereby calculating the signal x′ when the expression 800 is applied for the patient i. The signal x′ of the patient i is referred to as a signal xᵢ′. The signal x′ is a calculated value of the expression 800. In the first analysis target data 210 of FIG. 2, since the number of patients (total number of patient IDs 201) is 50, the number of signals x′ is 50.

The signal x′ is stored in the data memory 400 and output to the controller 404. When the expression 800 cannot be configured from the sequence data indicated by the action history row 802, the modulator 401 sets the values of all the signals x′ to 0. Accordingly, step S901 ends, and the process proceeds to step S902.

### Step S902

The modulator 401 sets the stop signal K(t) to K(t)=1 when all the columns in the action history row 802 are filled (i.e., the time step t=T−1) or when “End” is selected as the modulation method, and otherwise sets K(t)=0. Accordingly, step S902 ends, and the process proceeds to step S903.

### Step S903

At the current time step t, the spectral generator 402 generates the multispectral signal S(t) serving as an identification signal from the signal x′ obtained in step S901. Specifically, for example, the spectral generator 402 calculates a signal position SP(t) by the following expression (1).

SP(t)=floor((d−1)(x′−min(x′)/(max(x′)−min(x′))) (1)

In a right side of the above expression (1), d (an integer of 0 or more) is a signal length of the spectral signal. min(x′) is an operation for selecting a minimum value in all signals x′, and max(x′) is an operation for selecting a maximum value in all signals x′. In addition, a function floor( ) is a function for truncating to an integer value.

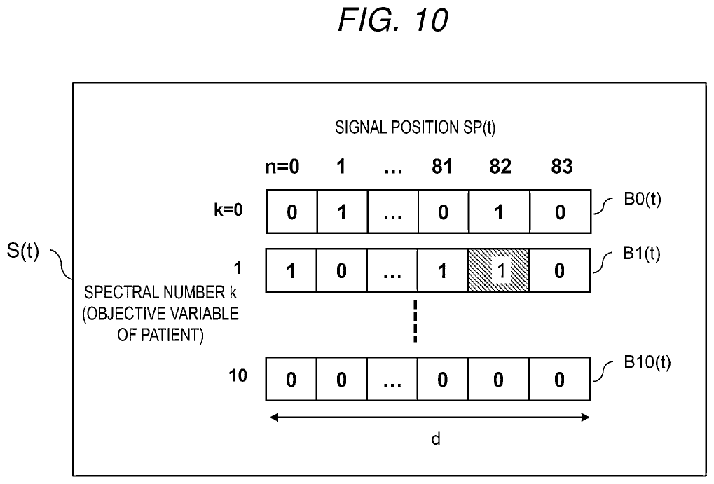

FIG. 10 is a diagram showing an example of the multispectral signal S(t) according to the first embodiment. The multispectral signal S(t) is a set of arrays Bk(t) of columns indicating spectral signals for each spectral number k. The spectral number k is a number for uniquely specifying the class to which the patient belongs. In FIG. 10, there are 11 classes from k=0 to 10. An array number n is an integer of d=0 to 83.

The multispectral signal S(t) is expressed as a matrix of (d+1)×(k+1). In FIG. 10, d=83 and k=10. The maximum value of the array number n is d−1.

In each column in the array Bk(t), a value indicating whether the value corresponds to the integer value output from the above expression (1) is set. “1” is set in a corresponding case, and “0” is set in a non-corresponding case. An initial value of the column is also “0”.

An example of a process of updating the value of the column in the array Bk(t) from “0” to “1” will be described. The spectral generator 402 applies the signal xᵢ′ of the patient i and the signals x′ of all the patients to the above expression (1) to calculate the signal position SP(t) of the patient i at the time step t, and specifies the array number n matching the calculated signal position SP(t).

The spectral generator 402 acquires a value of the objective variable 202 of the patient i from the first analysis target data 210, and specifies the spectral number k matching the acquired value. The spectral generator 402 updates the value of the column in the array Bk(t) corresponding to the specified array number n and the specified spectral number k from “0” to “1”.

For example, it is assumed that the specified array number n is n=82. When a value i of the patient ID 201 is i=1, k=1 because the value of the objective variable 202 is “1”. Therefore, for the patient i (i=1), “1” is set in the column with the array number n=82 in a hatched array B1(t). The same process is performed for the patients i (i=2 to 50) to generate the multispectral signal S(t) at the time step t. The multispectral signal S(t) is stored in the data memory 400 and output to the controller 404 at each time step t, and the subroutine 900 returns processing to the main routine 600.

### Step S603

Returning from the subroutine 900 to FIG. 6, the controller 404 determines the control signal a(t) at the time step t. Specifically, for example, the controller 404 outputs a random numerical value in a range of 0.0 to 1.0 by the random unit 503. When the random numerical value output from the random unit 503 is equal to or greater than a threshold value e (for example, e=0.5), the controller 404 randomly selects one action 302 from the pattern table 300, and determines the control signal a(t) by the selected action 302.

For example, when the action 302 randomly selected from the pattern table 300 at a certain time step t is “/” of the value “104” of the action number 301, the controller 404 determines “/” to be the control signal a(t).

On the other hand, when the random numerical value output by the random unit 503 is less than the threshold value e at a certain time step t, the controller 404 inputs the multispectral signal S(t) to the Q* network 501 in the network unit 500, and generates the value map z(t).

The controller 404 selects one action 302 corresponding to the action number 301 having the maximum value in the value map z(t) from the pattern table 300, and determines the selected action 302 to be the control signal a(t).

For example, in FIG. 3, the maximum value in the value map z(t) is “0.9” and corresponds to the action number 102. In the pattern table 300, the action 302 corresponding to the value “102” of the action number 301 is “exp”. The controller 404 determines the control signal a(t) to be “exp” corresponding to the maximum value “0.9”. By selecting the action 302 having the maximum value in this way, the controller 404 can select the control signal a(t) having a higher value, and the controller 404 can take a more suitable action 302.

### Step S604

The evaluator 403 calculates the reward r(t) at the time step t. Specifically, for example, in step S602, the evaluator 403 trains the identification model using the signal x′ output from the controller initialization subroutine 900 and the value of the objective variable 202 loaded from the data memory 400, and calculates the prediction accuracy.

As the identification model, a prediction model such as logistic regression, support vector machine (SVM), or gradient boost can be used. Regardless of which prediction model is used, the reward r(t) at the time step t can be calculated using the statistics V(t) (AUC, accuracy, precision, recall, f-measure, etc.) with which whether the identification is correctly performed can be known. In the first embodiment, logistic regression, which is the simplest configuration, will be described as an example.

p=model(x′) (2)

V(t)=score(p,target) (3)

To explain using the above expression (2), the evaluator 403 inputs the signal x′ to the learned identification model (the logistic regression model in the first embodiment) to calculate a predicted value p. Next, as shown in the above expression (3), the evaluator 403 substitutes the predicted value p and the objective variable 202 (represented as “target” in the expression (3)) into a score function score( ) to calculate the statistic V(t) at a certain time step t.

In the first embodiment, as shown in FIG. 7, in step S600, although the user selects “AUC” as the score function score( ) in the statistic selection unit (Measure) 741, the above expression (2) can be similarly configured as long as the statistic V(t) is a statistic such as f-measure that can evaluate the prediction accuracy of the identification model.

Then, the evaluator 403 calculates the reward r(t) at the time step t using the statistic V(t) by the following expression (4). The following expression (4) is configured as a calculation expression for the reward r(t) so that the doctor or the researcher intuitively feels that excellent identification has been made from the signal x′.

r(t)=V(t)+(1−Overwrap)+Margin (4)

Overwrap on a right side of the above expression (4) is a ratio at which points of different classes overlap each other. Taking the signal distribution 760 of FIG. 7 as an example, none of the points in the point group 761 of the class 0 overlaps with any points in the point group 762 of the class 1. Therefore, the points of different classes do not overlap at all. In this case, the rate at which points of different classes overlap each other is 0.

Taking FIG. 10 as an example, when an array B0(t) and the array B1(t) are compared, the column with the array number n=82 is “1” in both. Therefore, the point of the class 0 (k=0) and the point of the class 1 (k=1) overlap each other in the signal distribution 760. Assuming that an overlapping position is this one place, the ratio at which points of different classes overlap each other is 1/84 since the signal length d=84.

Margin on a right side of the above expression (4) is a width between different classes. Taking the signal distribution 760 of FIG. 7 as an example, the margin 763 indicating the interval between the boundary point 761L and the boundary point 762R is Margin. Taking FIG. 10 as an example, when the array B0(t) and the array B1(t) are compared, points of different classes overlap each other since the value of the array number n=82 is “1” in both. Therefore, Margin=0.

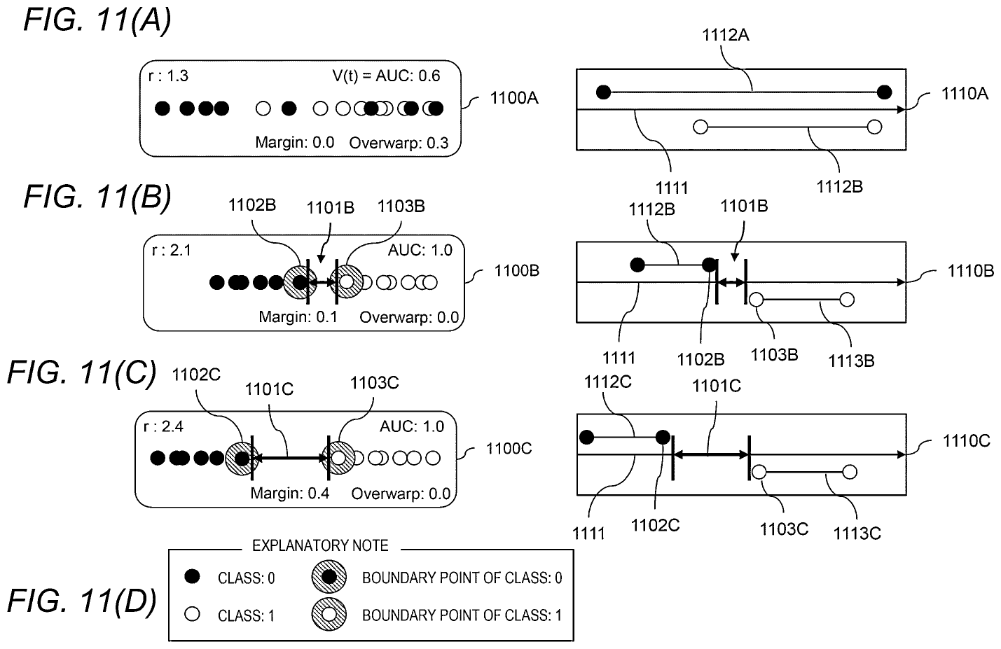

When the number of classes is larger than 2 (k≥2), Overwrap and Margin are calculated in a round-robin manner between different classes and added to the above expression (4). Here, a calculation example of Margin will be specifically described with reference to FIGS. 11(A) to 11(C).

FIGS. 11(A) to 11(C) are diagrams showing a calculation example of Overwrap and Margin according to the first embodiment. FIG. 11(A) shows a signal distribution 1100A and a panel 1110A indicating a class distribution thereof. FIG. 11(B) shows a signal distribution 1100B and a panel 1110B indicating a class distribution thereof. FIG. 11(C) shows a signal distribution 1100C and a panel 1110C indicating a class distribution thereof.

When the signal distributions 1100A, 1100B, and 1100C are not distinguished from one another, they are referred to as a signal distribution 1100. When the panels 1110A, 1110B, and 1110C are not distinguished from one another, they are referred to as a panel 1110. In addition, each point (• and ∘) of the panel 1110 is the signal x′ of each patient, i.e., the calculated value of the generation equation 770.

The signal distribution 1100 is output from the output device 104 in a displayable manner, and data related to the signal distribution 1100 is transmitted to another computer via the communication IF 105, so that the signal distribution 1100 is output in the other computer in a displayable manner. The panel 1110 is internal processing data, but may be output together with the signal distribution 1100 or instead of the signal distribution 1100 in a displayable manner.

(A) In the signal distribution 1100A, the distribution of the point group (•) of the class 0 and the distribution of the point group (∘) of the class 1 overlap each other. In the signal distribution 1100A, 25 points overlap between class 0 and class 1 since the value of Overwrap is 0.3. Since the distribution of the point group (•) of the class 0 and the distribution of the point group (∘) of the class 1 overlap each other, the value of Margin is 0.

The panel 1110A includes a number straight line 1111, a distribution range 1112A of the point group of the class 0, and a distribution range 1113A of the point group of the class 1. A black circle at a left end of the distribution range 1112A of the point group of the class 0 is a point where the signal x′ is minimum in the class 0, and a black circle at a right end thereof is a point where the signal x′ is maximum in the class 0. Similarly, a white circle at a left end of the distribution range 1113A of the point group of the class 1 is a point where the signal x′ is minimum in the class 1, and a white circle at a right end thereof is a point where the signal x′ is maximum in the class 1.

(B) In the signal distribution 1100B, the distribution of the point group (•) of the class 0 and the distribution of the point group (∘) of the class 1 do not overlap (Overwrap=0), and the signal distribution 1100B has a margin 1101B (Margin>0). The margin 1101B is an interval between a point 1102B where the signal x′ is maximum among the point group of the class 0 and a point 1103B where the signal x′ is minimum among the point group of the class 1. That is, the margin 1101B is a value obtained by subtracting the signal x′ indicating the position of the point 1102B from the signal x′ indicating the position of the point 1103B.

The panel 1110B includes a number straight line 1111, a distribution range 1112B of the point group of the class 0, and a distribution range 1113B of the point group of the class 1. A black circle at a left end of the distribution range 1112B of the point group of the class 0 is a point where the signal x′ is minimum in the class 0, and a black circle at a right end thereof is a point where the signal x′ is maximum in the class 0. Similarly, a white circle at a left end of the distribution range 1113B of the point group of the class 1 is a point where the signal x′ is minimum in the class 1, and a white circle at a right end thereof is a point where the signal x′ is maximum in the class 1.

(C) In the signal distribution 1100C, the distribution of the point group (•) of the class 0 and the distribution of the point group (∘) of the class 1 do not overlap (Overwrap=0), and the signal distribution 1100C has a margin 1101C (Margin>0). The margin 1101C is an interval between a point 1102C where the signal x′ is maximum among the point group of the class 0 and a point 1103C where the signal x′ is minimum among the point group of the class 1.

The panel 1110C includes a number straight line 1111, a distribution range 1112C of the point group of the class 0, and a distribution range 1113C of the point group of the class 1. A black circle at a left end of the distribution range 1112C of the point group of the class 0 is a point where the signal x′ is minimum in the class 0, and a black circle at a right end thereof is a point where the signal x′ is the maximum in the class 0. Similarly, a white circle at a left end of the distribution range 1113C of the point group of the class 1 is a point where the signal x′ is minimum in the class 1, and a white circle at a right end thereof is a point where the signal x′ is maximum in the class 1.

Thus, the evaluator 403 calculates the reward r(t) by substituting the statistic V(t) calculated by the above expression (3) and the calculated Overwrap and Margin into the above expression (4). In FIGS. 11(A) to 11(C), an example of two-class classification has been described, but in the case of three or more classes, (1−Overwrap) and Margin calculated from all combinations between classes are substituted into the above expression (4).

The reward r(t) calculated by the above expression (4) increases as the number of corresponding conditions increases among three conditions including (a) the prediction accuracy is high due to the statistic V(t), (b) the points of different classes do not overlap each other (i.e., the value of (1−Overwrap) is large), and (c) the points of different classes are distributed away from each other (the value of Margin is large).

In FIG. 11(A), the prediction accuracy (AUC) is as low as V(t)=0.6, 30% of points overlap between the different classes, and the margin indicating the distance between the classes is 0. Therefore, the reward r(t)=1.3.

In FIG. 11(B) and FIG. 11(C), the prediction accuracy (V(t)=1.0) is equal, and there is no overlap between the classes. On the other hand, the margin indicating the distance between the classes is larger in FIG. 11(C), and the reward r(t) in FIG. 11(C) is higher by 0.3 points (=2.4 to 2.1). The evaluator 403 stores the reward r(t) in the data memory 400 and outputs the reward r(t) to the controller 404.

### Step S605

The signal processing apparatus 100 executes signal data generation processing at a time step t+1 shown in FIG. 8. Specifically, for example, the signal processing apparatus 100 calculates the multispectral signal S(t+1) and the signal x′ by the subroutine 900.

### Step S606

The network unit 500 stores the reward r(t), the multispectral signals S(t) and S(t+1), the control signal a(t), and the stop signal K(t) as the data pack D(t) in the replay memory 411 in the data memory 400.

### Step S607

When the stop signal K(t)=0 (step S607: Yes), the signal processing apparatus 100 updates the time step t as t=t+1, and returns to step S603. On the other hand, when the stop signal K(t)=1 (step S607: No), the signal processing apparatus 100 shifts the process to step S608.

### Step S608

The training parameter update unit 520 loads J data packs D(1), . . . , D(j), . . . , and D(J) (j=1, . . . , and J) (hereinafter referred to as a data pack group Ds) from the replay memory 411 at random, and updates a teacher signal y(j) by the following expression (5). In the first embodiment, J=100 as an example.

$$y ( j ) = \{ \begin{matrix}r ( j ) , & if K ( j ) = 1 \\ r ( j ) + γ max Q ( I ( j + 1 ) ; θ ) , & otherwise\end{matrix} \qquad ( 5 )$$

In the above expression (5), γ is a discount rate, and in the first embodiment, γ=0.998. A calculation process maxQ(S(j+1); θ) in the above expression (5) is a process in which a multispectral signal S(j+1) is input to the Q network 502 in the network unit 500, and the Q network 502 outputs the maximum value, i.e., the maximum action value from a value map z(j) calculated by applying the training parameter θ. For example, when the value map z(t) in FIG. 3 is the value map z(j), the calculation process maxQ(S(j+1); θ) outputs the value “0.9” of the action number=102 as the maximum action value.

### Step S609

The training parameter update unit 520 executes learning calculation. The gradient calculation unit 521 updates the training parameter θ by outputting a gradient for the training parameter θ using the following expression (6).

θ=θ+αgrad_{θ}(y(j)−Q(I(j);θ))² (6)

A second term grade on a right side of the above expression (6) is a function for calculating the gradient for the training parameter θ. α is a training coefficient having a positive real value (in the first embodiment, α=0.001 as an example). Accordingly, the Q network 502 can generate, by using the updated training parameter θ that takes into account the reward r(t), the reward r(t), i.e., the control signal a(t) indicating the action 302 that increases the prediction accuracy of the objective variable.

In step S609, the training parameter update unit 520 overwrites the training parameter θ* of the Q* network 501 with the updated training parameter θ of the Q network 502. That is, the Q* network 501 has a value same as that of the updated training parameter θ. Accordingly, the Q* network 501 can specify the control signal a(t) as an action which can be expected to increase the action value, i.e., the prediction accuracy of the objective variable.

### Step S610

When the statistic V(t) is less than the target value input to the target value setting unit 742 and the number of calculation steps m is less than the predetermined number of times M (step S610: Yes), the signal processing apparatus 100 returns to step S602 and updates the calculation step m to m=m+1 in order to continue the analysis by the signal processing apparatus 100. In the first embodiment, M=1,000,000 times as an example.

On the other hand, when the statistic V(t) is equal to or greater than the target value input to the target value setting unit 742 or the number of calculation steps m reaches the predetermined number of times M (step S610: No), the signal processing apparatus 100 proceeds to step S611.

### Step S611

The signal processing apparatus 100 stores, in the storage device 102, an action history A(m′) of all calculation steps m′=1, . . . , and M′ with the statistic V(t) equal to or greater than the target value, and a data pack D(t≤t′) at a time step equal to or less than a time step t′ at the calculation step m′, among the data pack group Ds stored in the data memory 400.

### Step S612

The signal processing apparatus 100 executes a result display as an output unit. Specifically, for example, the signal processing circuit 107 outputs the final signal distribution 760 and the generation equation 770 from a plurality of action histories A(m′) and the data pack D(t≤t′) at the time step equal to or less than the time step t′ associated with the calculation step m′, which are stored in the storage device 102. The processor 101 displays, as an output unit, the final signal distribution 760 and the generation equation 770 output from the signal processing circuit 107 in the result display area 750. Accordingly, all the processes of the main routine 600 end.

The signal x′ generated as described above and the generation equation 770 thereof are easy for the doctor and the researcher to consider the results in a medical manner and determine the effect of the drug and the like. Therefore, it is possible to assist in searching for the mechanism through the generation equation 770. In addition, by handling the multispectral signal S(t), it is possible to reduce the memory amount required for the calculation process and to contribute to speeding up the calculation process.

### Experiment

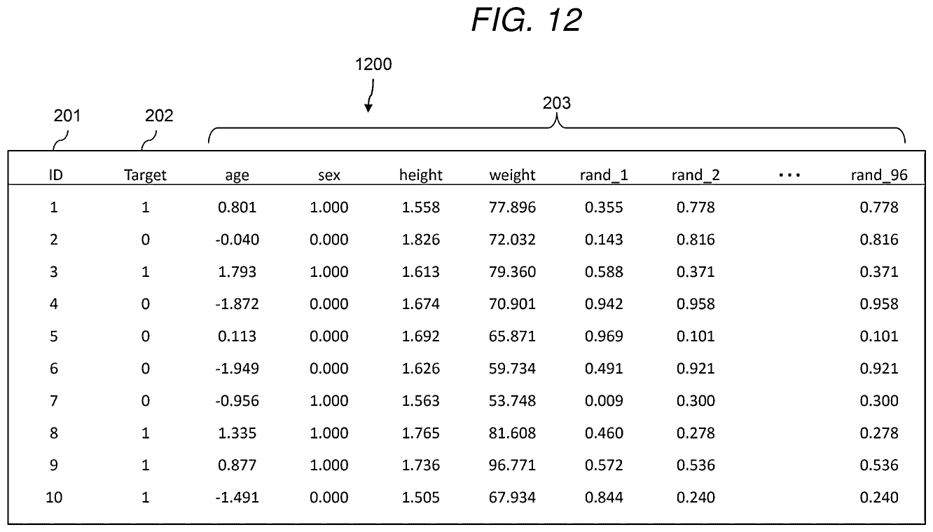

FIG. 12 is a diagram showing an example of patient data used in an operational experiment of the signal processing apparatus 100 according to the first embodiment. Patient data 1200 is a specific example of the first analysis target data 210. Here, operation results of the signal processing apparatus 100 according to the first embodiment will be described. The patient data 1200 in FIG. 12 is an excerpt of the patient data used in the experiment.

The number of patients is 442 (10 in FIG. 12 because the patient ID 201 is 1 to 10), and the explanatory variable group 203 includes an age, a sex, a height, a weight, and a total of 100 kinds of 96 uniform random numbers. Age values are normalized to mean 0 and variance 1. For a sex value, “0” represents a female, and “1” represents a male. Target (objective variable) is set to “1” when BMI calculated from weight/height² is larger than a median value of BMI of all patients, and is set to “0” otherwise.

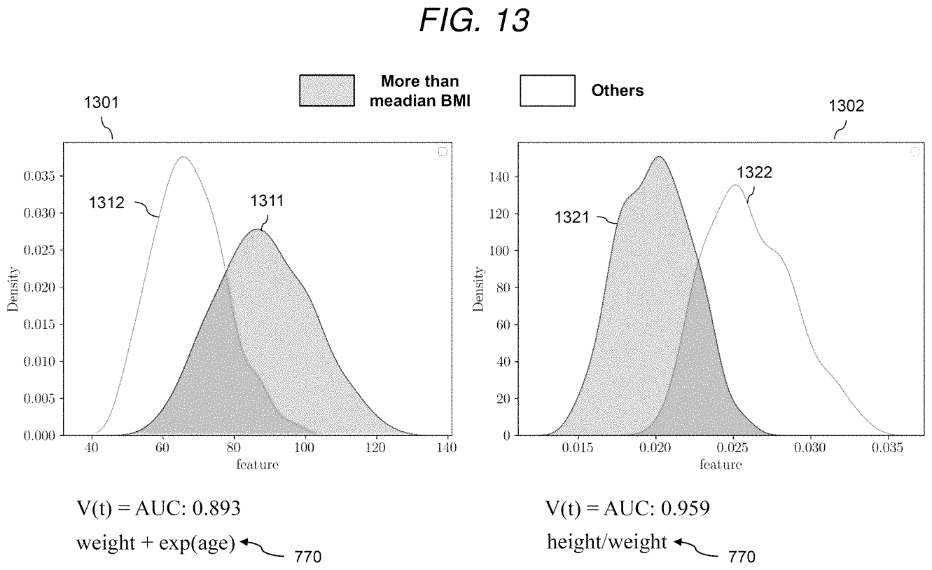

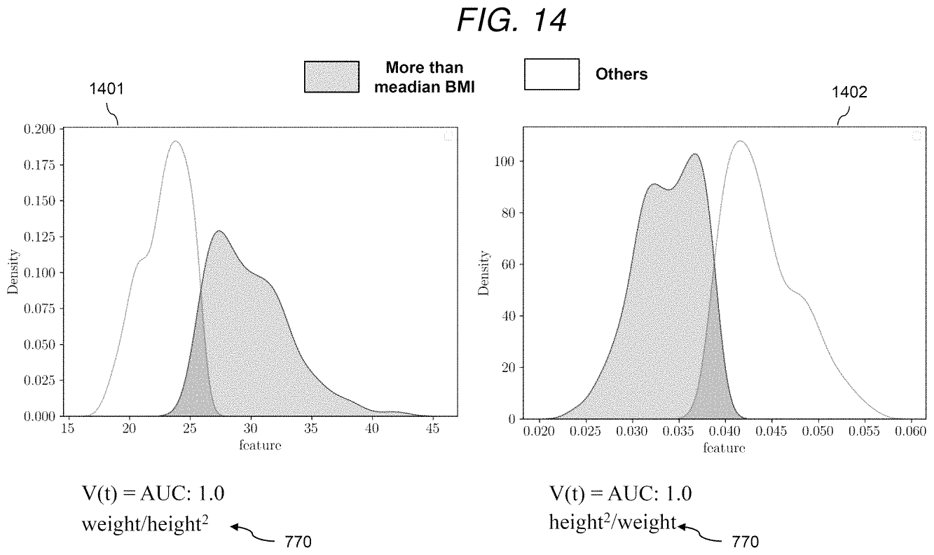

FIG. 13 is a graph showing a first example of an operation experiment result of the signal processing apparatus according to the first embodiment, and FIG. 14 is a graph showing a second example of the operation experiment result of the signal processing apparatus according to the first embodiment. In FIGS. 13 and 14, panels 1301, 1302, 1401, and 1402 illustrate distributions of values of signals x′ of the patients using kernel density estimation. A horizontal axis represents the value of the signal x′, and a vertical axis represents a kernel density estimation amount (approximately, frequency).

As a result of operating the signal processing apparatus 100,
regarding the panel 1301,
statistic V(t)=AUC: 0.893
generation equation 770: weight+exp(age),
   regarding the panel 1302,
statistic V(t)=AUC: 0.959
generation equation 770: height/weight,
regarding the panel 1401,
statistic V(t)=AUC: 1.0
generation equation 770: weight/height², and
   regarding the panel 1402,
statistic V(t)=AUC: 1.0
generation equation 770: height²/weight.

The statistic V(t) of the panel 1401 is AUC: 1.0, and the BMI is correctly restored from the value on the horizontal axis. Next, the height²/weight of the generation equation 770, which is the result of the panel 1402, is a reciprocal of the BMI, and can be handled in the same manner as the BMI in the case of the application for stratification. In this manner, the doctor or the researcher can determine medical validity through the generation equation. From the above results, it was confirmed that the configuration according to the first embodiment can perform stratification as intended.

### Second Embodiment

A second embodiment is an example of applying the second analysis target data 220 instead of the first analysis target data 210 shown in FIG. 2 in the first embodiment. A difference from the first analysis target data 210 is that the objective variable 202 of the first analysis target data 210 is a qualitative variable, whereas an objective variable 212 of the second analysis target data 220 is a quantitative variable. In the second embodiment, differences from the first embodiment will be mainly described, so that the same components as those of the first embodiment are denoted by the same reference numerals, and the description thereof will be omitted.

### Example of Processing Procedure

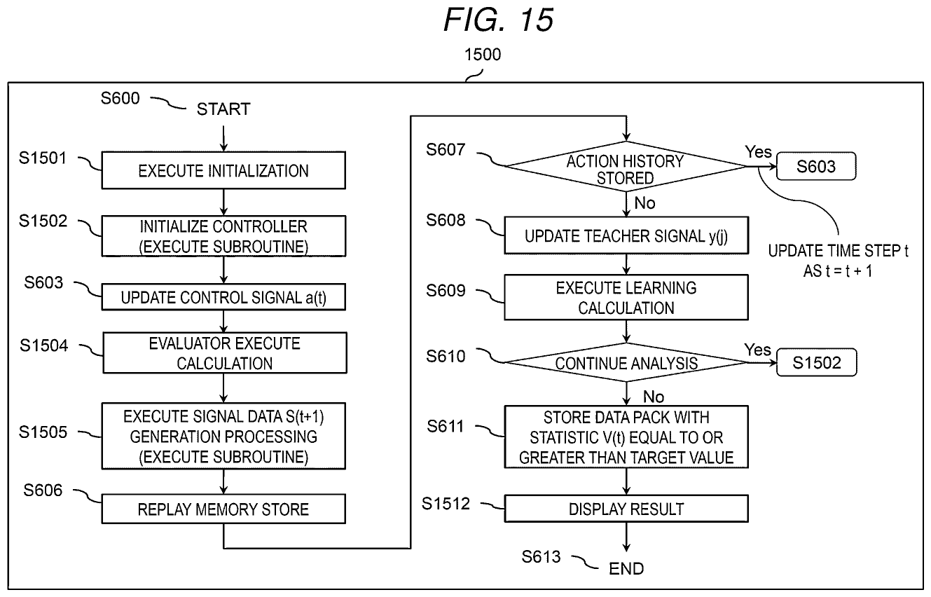

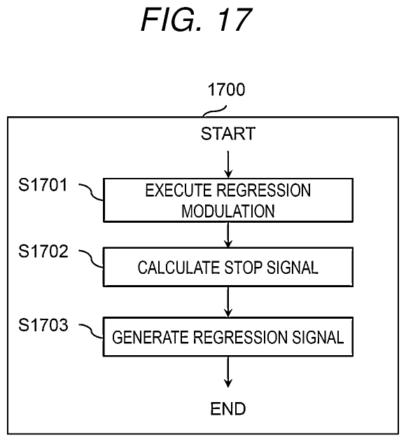

FIG. 15 is a flowchart showing a main routine 1500 as an example of a processing procedure performed by the signal processing apparatus 100 according to the second embodiment. Hereinafter, a flow of the processing of the main routine 1500 will be described with reference to the flowchart of FIG. 15. In the second embodiment, a subroutine shown in FIG. 17 is executed instead of the subroutine 900.

### Step S1500

A display screen is displayed on the output device 104.

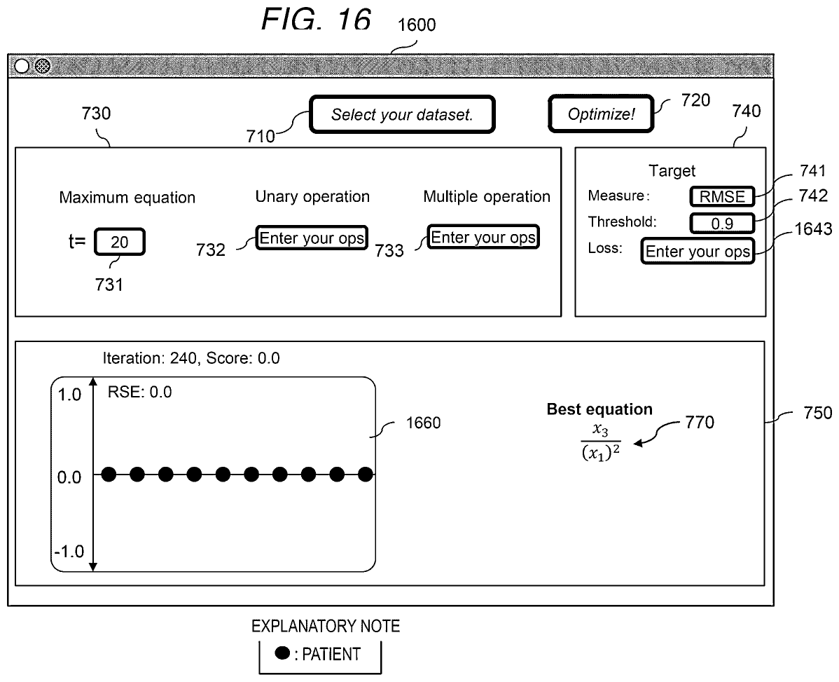

FIG. 16 is a diagram showing an example of the display screen according to the second embodiment. A display screen 1600 includes the load button 710, the start button 720, the generation condition input area 730, the target scale input area 740, and the result display area 750. When a user clicks the load button 710, the second analysis target data 220 in the analysis target DB 121 and the pattern table 300 in the pattern DB 122, which are stored in the storage device 102, are loaded using a function of an operation system. The processor 101 transfers the second analysis target data 220 and the pattern table 300 to the data memory 400 of the signal processing circuit 107. When the user clicks the start button 720, processing of the main routine 1500 is started.

A difference from the first embodiment is that, in the second embodiment, in order to generate the multispectral signal S(t) representing a quantitative variable, a relative squared error (RSE) is input to the statistic selection unit 741, and “0.9” is set in the target value setting unit 742. The statistic selection unit 741 can also select other statistics (a square error, a relative absolute error, a determination coefficient, etc.) other than RSE, which can evaluate prediction accuracy of a regression model.

One or more loss functions for calculating the multispectral signal S(t) can be set in a loss function setting unit 1643 of the target scale input area 740. In the second embodiment, it is assumed that a signed square error of the following expression (7) is set.

P=sign(target−x′)(target−x′)² (7)

In addition, at least one of a signed mean absolute error (the following expression (8)) and a signed hinge error (the following expression (9)) can be set in the loss function setting unit 1643.

$$P = sign ( target - {x}^{′} ) \vert target - {x}^{′} \vert \qquad ( 8 )$$

$$P = \{ \begin{matrix}0 , & \vert target - {x}^{′} \vert < ε \\ sign ( target - {x}^{′} ) \vert target - {x}^{′} \vert , & otherwise\end{matrix} \qquad ( 9 )$$

A sign function of the above expressions (7) to (9) is a function for receiving a value and returning a sign, outputs “1.0” when an argument is equal to or greater than 0, and outputs “−1.0” when the argument is less than 0. In the above expression (9), ε is a parameter representing an allowable error, and is set to “0.1” in the second embodiment. A user may input an error function as an expression to the loss function setting unit 1643. For example, a signed logarithmic conversion hinge error function as shown in the following expression (10) can be input.

$$P = \{ \begin{matrix}0.1 sign ( target - {x}^{′} ) log \vert target - {x}^{′} \vert , & \vert target - {x}^{′} \vert < 1 \\ sign ( target - {x}^{′} ) log \vert target - {x}^{′} \vert , & othe r w i s e\end{matrix} \qquad ( 10 )$$

The result display area 750 includes a signal distribution 1660 and the generation equation 770. In the signal distribution 1660, a vertical axis represents a magnitude of loss (a value of P). In addition, a horizontal axis represents an index of the objective variable 212 (an output value of an argsort function of an expression (11) to be described later) when a magnitude of the objective variable 212 (target) is rearranged in ascending order. The signal distribution 1660 of FIG. 16 indicates that the loss function P=0 for each patient. That is, this means that the objective variable 212 of each patient and the signal x′ completely match.

### Step S1502

Returning to FIG. 15, after the execution of step S1501, the signal processing apparatus 100 executes initialization of the controller 404 as in step S602. However, in step S1502, the signal processing apparatus 100 executes a subroutine 1700 instead of the subroutine 900.

### Subroutine

FIG. 17 is a flowchart showing an example of a detailed processing procedure of the subroutine 1700 in the main routine 1500 in step S1502. The subroutine 1700 is called and executed by step S1502 and step S1505 of the main routine 1500.

### Step S1701

The modulator 401 executes regression modulation. Specifically, for example, the modulator 401 selects an explanatory variable or a modulation method from the control signal a(t) output from the controller 404 at the time step t. The modulator 401 may receive selection of the explanatory variable or the modulation method selected by the user.

The modulator 401 adds the selected variable or modulation method to the column in the action history row 802 at the time step t. An initial value of the action history row 802 is blank for all columns.

When reading out the sequence data indicated by the action history row 802 column by column in ascending order of the time step t, the modulator 401 generates an expression by a reverse polish notation. In the example of FIG. 8, the expression 800 is generated. In addition, the modulator 401 substitutes, into the expression 800, a value of the explanatory variable existing in the expression 800 from the explanatory variable group 203 of the patient i to calculate the signal x′ when the expression 800 is applied for the patient i. The signal x′ is a calculated value of the expression 800. In the second analysis target data 220 of FIG. 2, since the number of patients (total number of patient IDs 201) is 50, the number of signals x′ is 50.

The signal x′ is stored in the data memory 400 and output to the controller 404. When the expression 800 cannot be configured from sequence data indicated by the action history row 802, the modulator 401 sets the values of all signals x′ to 0. Accordingly, step S1701 ends, and the process proceeds to step S1702.

### Step S1702

The modulator 401 sets the stop signal K(t) to K(t)=1 when all the columns in the action history row 802 are filled (i.e., t=T−1) or when “End” is selected as the modulation method, and otherwise sets K(t)=0. Accordingly, step S1702 ends, and the process proceeds to step S1703.

### Step S1703

At the current time step t, the spectral generator 402 generates the multispectral signal S(t) from the signal x′ obtained in step S1701. Specifically, for example, the spectral generator 402 calculates the signal position SP(t) by the following expression (11).

SP(t)=floor((d−1)argsort(target)/N) (11)

In the above expression (11), N is a total number of patient IDs 201 (N=50 in the second embodiment). argsort is a function for outputting an index (an integer starting from 0) of the objective variable 212 when the magnitude of the objective variable 212 (target) is rearranged in ascending order. For example, assuming that target={0.1, 0.0, 1}, argsort (target)={1, 0, 2} since the index of “0.1” is “1”, the index of “0.0” is “0”, and the index of “1” is “2”.

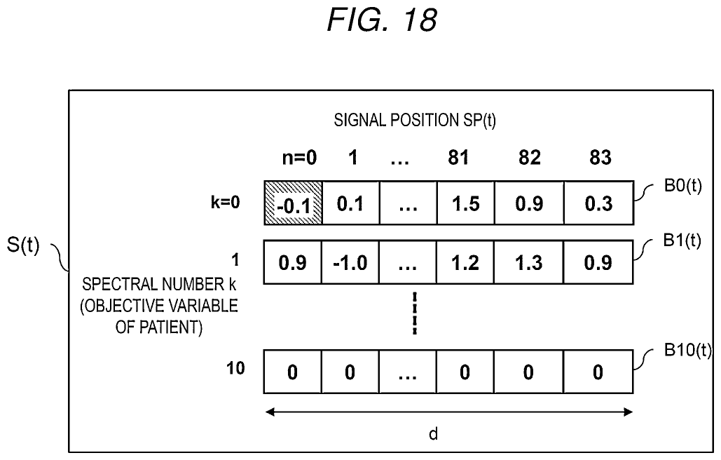

FIG. 18 is a diagram showing an example of the multispectral signal S(t) according to the second embodiment. The multispectral signal S(t) is a set of arrays Bk(t) of columns for each spectral number k. The spectral number k is a value of the objective variable 202 of the patient. In FIG. 10, there are 11 classes from k=0 to 10. An array number n is an integer of d=0 to 83. In the first embodiment, the value set in the column is “0” (initial value) or “1”, whereas in the second embodiment, the value set in the column is “0” (initial value) or a calculation result of the loss function set in the loss function setting unit 1643.

The spectral generator 402 calculates the multispectral signal S (t) using the above expression (7). For example, when the signal position SP(t)=0 in the above expression (11) and the loss function P=−0.1 in the above expression (7), the loss function P=−0.1 is set in the column with the array number n=SP(t)=0 in the array B0(t) with the spectral number k=0.

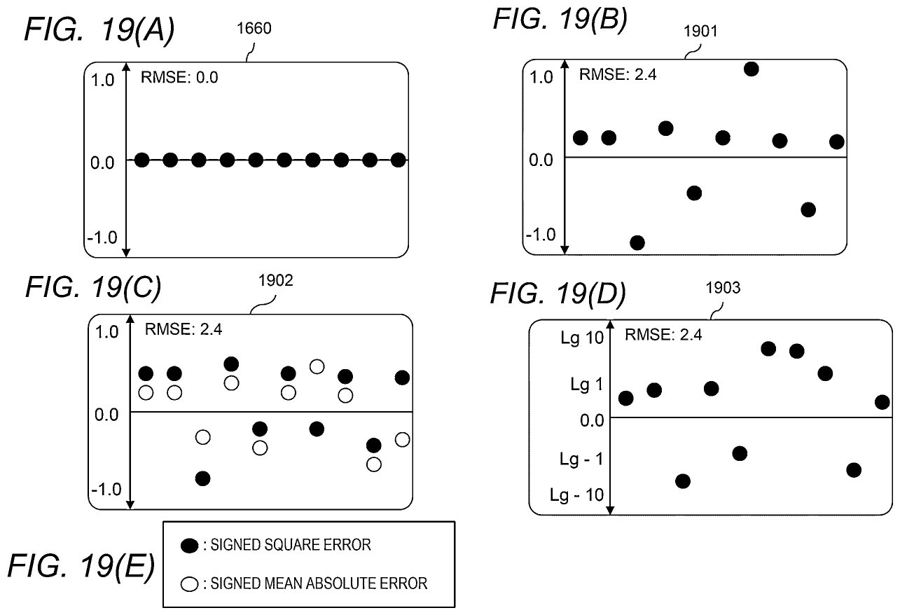

FIG. 19 is a diagram showing a visualization example of the multispectral signal S(t) according to the second embodiment. FIG. 19(A) shows the signal distribution 1660 shown in FIG. 16. FIG. 19(B) shows a signal distribution 1901 with a loss (P≠0) in the signal xᵢ′ for each patient i.

In the loss function setting unit 1643, when the signed square error (the above expression (7)) and the signed mean absolute error (the above expression (8)) are input, that is, when a plurality of loss functions are input, the spectral generator 402 assigns the spectral number k in input order of the loss functions and executes the calculation of the loss function P. The multispectral signal S(t) retains data as shown in FIG. 18 for each loss function.

FIG. 19(C) shows a signal distribution 1902 when the multispectral signal S(t) is stored for each loss function P. Specifically, for example, the signal xᵢ′ of the patient i is displayed as a black circle (•) for the signed square error (the above expression (7)) which is a first input to the loss function setting unit 1643, and is displayed as a white circle (∘) for the signed mean absolute error (the above expression (8)) which is a second input to the loss function setting unit 1643.

FIG. 19(D) shows a signal distribution 1903 when the error function (for example, the above expression (10)) input by the user to the loss function setting unit 1643 is applied. For example, when the loss function P has a logarithm as in the above expression (10), a vertical axis of the signal distribution 1903 is displayed on a logarithmic scale. The multispectral signal S(t) is stored in the data memory 400 and output to the controller 404, the subroutine 1700 returns processing to the main routine 1500, and the process returns to step S603.

### Step S1504

Returning to FIG. 15, after the execution of step S603, the evaluator 403 executes the calculation of the reward r(t) at the time step t. However, in the second embodiment, the evaluator 403 calculates the reward r(t) at the time step t, which is different from that in the first embodiment. Specifically, for example, in step S602, the evaluator 403 trains a regression model using the signal x′ output from the controller initialization subroutine 900 and the value of the objective variable 202 loaded from the data memory 400, and calculates the prediction accuracy.

As the regression model, linear regression, SVM regression, or gradient boost regression can be used. Regardless of which prediction model is used, statistics (a relative square error (RSE), a square error, a determination coefficient, etc.) with which how correctly the regression is performed can be known. In the second embodiment, a linear regression model having the simplest configuration will be described as an example.

The reward r(t) is configured by the following expression (12) so that a doctor or a researcher intuitively feels that excellent identification has been made from the signal x′.

r(t)=1/(1−V(t)) (12)

The reward r(t) calculated by the above expression (12) is designed to increase as the relative squared error (RSE) decreases. In the expression (12), it is assumed that the user selects the relative square error (RSE) as the prediction accuracy by the statistic selection unit 741. In the case of the determination coefficient, the above expression (4) is adopted, but in the case of application to the second embodiment, values of Overwrap and Margin in the above expression (4) are set to 0.

### Step S1505

The signal processing apparatus 100 executes signal data generation processing at a time step t+1 shown in FIG. 8. Specifically, for example, the signal processing apparatus 100 calculates the multispectral signal S(t+1) and the signal x′ by the subroutine 1700.

### Step S1512

After steps S606 to S611 are executed in the same manner as in the first embodiment, the signal processing apparatus 100 causes the signal processing circuit 107 to operate based on a plurality of action histories A(m′) and a data pack D(t≤t′) at a time step equal to or less than the time step t′ associated with the calculation step m′, which are stored in the storage device 102, thereby displaying the final signal distribution and generation equation 770 as shown in FIG. 19 on the result display area 750 and ending the all processing of the main routine 1500.

According to the second embodiment, the signal x generated as described above and the generation equation 770 thereof are easy for the doctor and the researcher to consider the results in a medical manner and determine the effect of the drug and the like. Therefore, it is possible to assist in searching for the mechanism through the generation equation 770. In addition, by handling the multispectral signal S(t), it is possible to reduce the memory amount required for the calculation process and to contribute to speeding up the calculation process.

The signal processing apparatus 100 according to the first embodiment and the second embodiment described above can also be configured as described in (1) to (13) below.

(1) The signal processing apparatus 100 includes: a storage unit (storage device 102) configured to store an analysis target data group (first analysis target data 210 or second analysis target data 220) including, for each analysis target (patient), analysis target data that includes a value of an explanatory variable of an explanatory variable group 203 and a value of an objective variable 202 for the analysis target, and action history information 412 retaining one or more actions 302 which are either the explanatory variable or a modulation method for modulating the explanatory variable; a modulator 401 which is a modulation unit configured to generate, based on the action history information, a first signal obtained by modulating the analysis target data for each analysis target; a spectral generator 402 which is a generation unit configured to generate a first multispectral signal S(t) obtained by classifying the first signal x′ modulated by the modulation unit for each analysis target into a first spectral signal for each value of the objective variable 202; and an output unit configured to generate, based on the first multispectral signal S(t), a signal distribution (760, 1100, 1660, 1901 to 1903) obtained by one-dimensionally arranging a distribution of the first signal x′ based on the value of the objective variable 202, and output the signal distribution in a displayable manner.

(2) In the signal processing apparatus 100 according to the above (1), the modulation unit combines the actions in the action history information to create an expression 800, acquires the value of the explanatory variable included in the expression 800 from the analysis target data, and outputs the first signal x′ that is a calculation result of the expression 800 for each analysis target.

(3) In the signal processing apparatus 100 according to the above (1), the storage unit stores a pattern table 300 including one or more explanatory variables and one or more modulation methods, and the signal processing apparatus 100 further includes a controller 404 that is a control unit configured to select a first action from the pattern table 300 and adds the first action to the action history information 412.

(4) In the signal processing apparatus 100 according to the above (1), the control unit randomly selects the first action from the pattern table 300.

(5) In the signal processing apparatus 100 according to the above (3), the control unit generates, based on a training parameter θ* and the first multispectral signal S(t), a first array (value map z(t)) indicating a value for each action, selects the first action corresponding to a specific value in the first array (value map z(t)), and adds the first action to the action history information 412.

(6) The signal processing apparatus 100 according to the above (3) further includes an evaluator 403 that is an evaluation unit configured to generate a training model based on the first signal x′ for each analysis target and the value of the objective variable 202, calculate a predicted value p for each analysis target by inputting the first signal x′ for each analysis target to the training model, and calculate, based on the predicted value p for each analysis target and the value of the objective variable 202, a reward r(t) for evaluating the value of the first action, in which the modulation unit generates, based on action history information 412 to which the first action is added by the control unit, a second signal x′ obtained by modulating the analysis target data for each analysis target (step S901), the generation unit generates a second multispectral signal S(t+1) obtained by classifying the second signal x′ modulated by the modulation unit for each analysis target into a second spectral signal based on the value of the objective variable 202 (step S903), and the control unit generates, based on the reward r(t), a training parameter θ, and the second multispectral signal S(t+1), a second array (value map z (j)) indicating a value for each action, selects a specific value in the second array (value map z(j)) (for example, selects a value “0.9” of an action number=102 as the maximum action value) (step S608), and updates the training parameter θ (step S609).

(7) In the signal processing apparatus 100 according to the above (6), the reward r(t) increases as prediction accuracy of the training model increases.

(8) In the signal processing apparatus 100 according to the above (6), the value of the objective variable 202 is an identification value related to the analysis target, the output unit generates, based on the first multispectral signal S(t), the signal distribution (760, 1100) obtained by one-dimensionally arranging a plurality of distributions of the first signal x′ for each value of the objective variable 202, and outputs the signal distribution in a displayable manner, and the reward r(t) increases as the number of overlapping portions of the plurality of distributions decreases.

(9) In the signal processing apparatus 100 according to the above (6), the value of the objective variable 202 is an identification value related to the analysis target, the output unit generates, based on the first multispectral signal S(t), the signal distribution (760, 1100) obtained by one-dimensionally arranging a plurality of distributions of the first signal x′ for each value of the objective variable 202, and outputs the signal distribution in a displayable manner, and the reward r(t) increases as an interval between the plurality of distributions increases.

(10) In the signal processing apparatus 100 according to the above (8), the output unit outputs the interval between the plurality of distributions in a displayable manner.

(11) In the signal processing apparatus 100 according to the above (1), the value of the objective variable 202 is a predicted value indicating a regression result related to the analysis target, the generation unit calculates a loss function P for each analysis target based on the value of the objective variable 202 and the first signal x modulated by the modulation unit for each analysis target, and generates a first multispectral signal S(t) obtained by classifying a calculation result of the loss function P according to the value of the objective variable 202, and the output unit generates, based on the first multispectral signal S(t), the signal distribution (1660, 1901 to 1903) indicating the calculation result of the loss function P for the first signal x′ arranged in order of the value of the objective variable 202, and outputs the signal distribution in a displayable manner.

(12) In the signal processing apparatus 100 according to the above (11), the generation unit generates the first multispectral signal S(t) for each loss function P when a plurality of the loss functions P are set, and the output unit generates, based on the first multispectral signal S(t) for each loss function P, one signal distribution 1902 including calculation results of the plurality of loss functions P for the first signal x′ arranged in order of the value of the objective variable 202, and outputs the signal distribution 1902 in a displayable manner.

It should be noted that the invention is not limited to the above-mentioned embodiments, and includes various modifications and the equivalent configurations within the gist of the scope of the appended claims. For example, the above-mentioned embodiment is described in detail in order to make the invention easy to understand, and the invention is not necessarily limited to those including all the configurations described above. In addition, a part of the configurations according to a given embodiment may be replaced with configurations according to another embodiment. A configuration of another embodiment can be added to a configuration of a certain embodiment. Further, a part of a configuration of each embodiment may be added to, deleted from, or replaced with another configuration.

Further, a part or all of the configurations, functions, processing units, processing means described above and the like may be implemented by hardware, for example by designing with an integrated circuit, or may be implemented by software, with a processor interpreting and executing a program that implements each function.

Information of a program, a table, and a file that implements each function can be stored in a storage apparatus such as a memory, a hard disk, and a solid state drive (SSD), or a recording medium such as an integrated circuit (IC) card, an SD card, and a digital versatile disc (DVD).

Control lines and information lines that are considered to be necessary for the description are shown, and not all the control lines and information lines that are necessary in terms of implementation are shown. It may be considered that almost all the configurations are actually connected to each other.

What is claimed is:

1. A signal processing apparatus comprising:
a storage unit configured to store an analysis target data group including, for each analysis target, analysis target data that includes a value of an explanatory variable and a value of an objective variable for the analysis target, and action history information retaining one or more actions which are either the explanatory variable or a modulation method for modulating the explanatory variable;
a modulation unit configured to generate, based on the action history information, a first signal obtained by modulating the analysis target data for each analysis target;
a generation unit configured to generate a first multispectral signal obtained by classifying the first signal modulated by the modulation unit for each analysis target into a first spectral signal for each value of the objective variable; and
an output unit configured to generate, based on the first multispectral signal, a signal distribution obtained by one-dimensionally arranging a distribution of the first signal based on the value of the objective variable, and output the signal distribution in a displayable manner.

2. The signal processing apparatus according to claim 1, wherein
the modulation unit combines the actions in the action history information to create an expression, acquires the value of the explanatory variable included in the expression from the analysis target data, and outputs the first signal that is a calculation result of the expression for each analysis target.

3. The signal processing apparatus according to claim 1, wherein
the storage unit stores pattern information including one or more explanatory variables and one or more modulation methods, and
the signal processing apparatus further comprises a control unit configured to select a first action from the pattern information, and add the first action to the action history information.

4. The signal processing apparatus according to claim 3, wherein
the control unit randomly selects the first action from the pattern information.

5. The signal processing apparatus according to claim 3, wherein
the control unit generates, based on a training parameter and the first multispectral signal, a first array indicating a value for each action, selects the first action corresponding to a specific value in the first array, and adds the first action to the action history information.

6. The signal processing apparatus according to claim 3, further comprising:
an evaluation unit configured to generate a training model based on the first signal for each analysis target and the value of the objective variable, calculate a predicted value for each analysis target by inputting the first signal for each analysis target to the training model, and calculate, based on the predicted value for each analysis target and the value of the objective variable, a reward for evaluating the value of the first action, wherein
the modulation unit generates, based on action history information to which the first action is added by the control unit, a second signal obtained by modulating the analysis target data for each analysis target,
the generation unit generates a second multispectral signal obtained by classifying the second signal modulated by the modulation unit for each analysis target into a second spectral signal based on the value of the objective variable, and
the control unit generates, based on the reward, a training parameter, and the second multispectral signal, a second array indicating a value for each action, selects a specific value in the second array, and updates the training parameter.

7. The signal processing apparatus according to claim 6, wherein
the reward increases as prediction accuracy of the training model increases.

8. The signal processing apparatus according to claim 6, wherein
the value of the objective variable is an identification value related to the analysis target,
the output unit generates, based on the first multispectral signal, the signal distribution obtained by one-dimensionally arranging a plurality of distributions of the first signal for each value of the objective variable, and outputs the signal distribution in a displayable manner, and
the reward increases as the number of overlapping portions of the plurality of distributions decreases.

9. The signal processing apparatus according to claim 6, wherein
the value of the objective variable is an identification value related to the analysis target,
the output unit generates, based on the first multispectral signal, the signal distribution obtained by one-dimensionally arranging a plurality of distributions of the first signal for each value of the objective variable, and outputs the signal distribution in a displayable manner, and
the reward increases as an interval between the plurality of distributions increases.

10. The signal processing apparatus according to claim 8, wherein
the output unit outputs the interval between the plurality of distributions in a displayable manner.

11. The signal processing apparatus according to claim 1, wherein
the value of the objective variable is a predicted value indicating a regression result related to the analysis target,
the generation unit calculates a loss function for each analysis target based on the value of the objective variable and the first signal modulated by the modulation unit for each analysis target, and generates the first multispectral signal obtained by classifying a calculation result of the loss function into a first spectral signal for each value of the objective variable, and
the output unit generates, based on the first multispectral signal, the signal distribution indicating the calculation result of the loss function for the first signal arranged in order of the value of the objective variable, and outputs the signal distribution in a displayable manner.

12. The signal processing apparatus according to claim 11, wherein
the generation unit generates the first multispectral signal for each loss function when a plurality of the loss functions are set, and
the output unit generates, based on the first multispectral signal for each loss function, one signal distribution including calculation results of the plurality of loss functions for the first signal arranged in order of the value of the objective variable, and outputs the signal distribution in a displayable manner.

13. A signal processing method executed by a signal processing apparatus storing an analysis target data group including, for each analysis target, analysis target data that includes a value of an explanatory variable and a value of an objective variable for the analysis target, and action history information retaining one or more actions which are either the explanatory variable or a modulation method for modulating the explanatory variable, the method comprising:
a modulation process of generating, based on the action history information, a first signal obtained by modulating the analysis target data for each analysis target;
a generation process of generating a first multispectral signal obtained by classifying the first signal modulated by the modulation process for each analysis target into a first spectral signal for each value of the objective variable; and
an output process of generating, based on the first multispectral signal, a signal distribution obtained by one-dimensionally arranging a distribution of the first signal based on the value of the objective variable, and outputting the signal distribution in a displayable manner.

14. A non-transitory computer readable medium storing a signal processing program, the signal processing program causing a computer storing an analysis target data group including, for each analysis target, analysis target data that includes a value of an explanatory variable and a value of an objective variable for the analysis target, and action history information retaining one or more actions which are either the explanatory variable or a modulation method for modulating the explanatory variable to execute:
a modulation process of generating, based on the action history information, a first signal obtained by modulating the analysis target data for each analysis target;
a generation process of generating a first multispectral signal obtained by classifying the first signal modulated by the modulation process for each analysis target into a first spectral signal for each value of the objective variable; and
an output process of generating, based on the first multispectral signal, a signal distribution obtained by one-dimensionally arranging a distribution of the first signal based on the value of the objective variable, and outputting the signal distribution in a displayable manner.
